(19) United States
(12) Patent Application Publication
(10) Pub. No.: US 2024/0400957 A1
(43) Pub. Date: Dec. 5, 2024

(54) HYBRID PHOTOBIOREACTOR

(71) Applicant: DEMETER BIOTECH (ZHUHAI) LTD., Zhuhai (CN)

(72) Inventors: Danxiang HAN, Zhuhai (CN); Liang ZHAO, Zhuhai (CN)

(21) Appl. No.: 18/804,149

(22) Filed: Aug. 14, 2024

Related U.S. Application Data
(63) Continuation of application No. PCT/CN2023/092147, filed on May 5, 2023.

(30) Foreign Application Priority Data
Mar. 16, 2022 (CN) 202210257049.X

(51) Int. Cl. C12M 1/00; C12M 1/02

(52) U.S. Cl. CPC C12M 21/02; C12M 29/08; C12M 29/26; C12M 31/08; C12M 31/10; C12M 41/18

(57) ABSTRACT

The present application relates to a hybrid photobioreactor, which includes a reactor body, wherein the reactor body is equipped with a water tank, a light source device, and a high-pressure rinsing and disinfecting device; the light source device includes transparent casings and LED light strips located within the transparent casings, and the transparent casings are arranged in parallel at intervals within the water tank; the high-pressure rinsing and disinfecting device comprises a plurality of horizontal tubes and a plurality of vertical tubes located under each horizontal tube; a plurality of liquid nozzles are provided on the wall of each vertical tube from top to bottom

### CROSS-REFERENCE TO RELATED APPLICATIONS

The present application is a continuation of International Application No. PCT/CN2023/092147, with an international filing date of May 5, 2023,which is based upon and claims priority to Chinese Patent Application No. 202210257049.X, filed on Mar. 16, 2022, the entire contents of all of which are incorporated herein by reference.

### TECHNICAL FIELD

The application relates to the technical field of microalgae's culture and production, and in particular to a hybrid photobioreactor.

### BACKGROUND

Microalgal biomass is rich in various high-value-added biological substances such as carbohydrates, proteins, fats, vitamins, pigments, bioactive substances, and trace elements, so it is becoming an important source of feedstocks for the broad applications in human food, medicine, dyes, and fine chemicals.

The key technology for large-scale culture of microalgae is photobioreactor. Currently, there are mainly three types of culture systems: open culture system, closed culture system and solid-state culture system. The open culture system was developed earlier and has been most widely used. However, it occupies a large area of land with low culture density, limited solar-energy utilization efficiency, and substantial water evaporation, and susceptibility to external environmental factors and vulnerability to contamination. Representative open photobioreactors include raceway culture ponds, circular culture ponds, etc. Compared with open culture system, the closed culture system has the advantages of: being less susceptible to contamination, high solar-energy utilization efficiency, easy temperature control, small water evaporation, and high cell density. However, the body of closed culture system is more expensive in terms of manufacturing, operation and maintenance, making large-scale application challenging. Representative closed photobioreactors include fermentation photobioreactors, horizontal or vertical tubular photobioreactors, etc., wherein the fermentation photobioreactors are mainly vertical fermentation tanks. For horizontal or vertical tubular photobioreactors, it is usually challenging for effective clean and disinfection, which jeopardizes the subsequent cultivation cycles.

There is also another type of solid-state culture system, in this system the algal strains are adhered onto the surface of porous material that can absorb and release water, then the algal strains are cultured by slowly supplying culture solution to the porous materials. However, such solid-state culture method depends significantly on the water retention properties of porous materials and the adhesion properties of algal strains. Therefore, if the material has poor water retention properties, it will require constant water supply, leading to high energy consumption. If the algal strain has poor adhesion property, it will easily detach, hindering normal growth. Furthermore, these porous materials are often opaque and absorb more solar energy, resulting in a lower light utilization efficiency for the algal strains, on the other hand, the porous structure also allows algal strains to enter the interior spaces of the materials and block the pores, causing difficulties to the subsequent culture solution supply, harvesting, cleaning and sterilization. Additionally, many of these materials will get mouldy and/or rotten after being used for culture once or twice. These solid-state culture methods are quite unsuitable for culturing algae with larger volume, such as Nostoc flagelliforme, Spirulina, Nostoc Sphaeroides and Nostoc commune.

In summary, the existing photobioreactors have the following technical problems: (1) Open raceway pond occupies expansive area with low culture density and high energy consumption for algal culture solution circulation, and it is susceptible to external contamination; (2) The manufacturing cost of the closed translucent container (especially glass container) is high, and due to the particularity of the glass processing technique, such container cannot be formed in one piece, resulting in exorbitant expenses for manufacturing, installation and maintenance. Additionally, there are further issues such as low cell density, inadequate space utilization, and high energy consumption; it is difficult to clean and disinfect the container after use, thus hindering convenient repeated production. (3) Solid-state culture photobioreactor is highly dependent on the inherent properties of materials, which has low light utilization efficiency and relatively limited applicability; further, its culture solution supply device is energy-consuming, so the culture cost cannot be further reduced. (4) The existing photobioreactors cannot be sterilized by high-temperature steam, hampering effective isolation and control of pollution sources. (5) The current photobioreactors generally rely either on natural light or artificial light sources alone, resulting in poor adaptability, and high energy consumption and manufacturing cost of pure artificial light sources.

### SUMMARY

In response to the above shortcomings, the purpose of the present application is to provide a hybrid photobioreactor that has low manufacturing cost, low energy consumption, and high light utilization efficiency, which can be cleaned and disinfected easily, and is suitable for large-scale industrialized culture of microalgae with broad applications, so as to reduce microalgae's production cost as a whole.

To achieve the above purpose, the primary technical solution of the present application includes:
the present application provides a hybrid photobioreactor, comprising:
a reactor body, wherein the reactor body is equipped with a water tank for microalgal cell culture, a light source device, and a high-pressure rinsing and disinfecting device;
the light source device comprises a plurality of transparent casings and LED light strips located within the transparent casings, and the transparent casings are arranged in parallel at intervals within the water tank;
the high-pressure rinsing and disinfecting device comprises a plurality of horizontal tubes and corresponding vertical tubes located beneath each horizontal tube, and each horizontal tube forms a comb-shaped structure, interconnected below by the vertical tubes; a plurality of liquid nozzles are provided on the wall of each vertical tube from top to bottom; the vertical tube has a lower end close to the bottom of the water tank; the plurality of horizontal tubes are connected to a general liquid inlet which is connected to a supply of high-pressure clean water, sterilizing water or sterilizing steam; the transparent casings and the horizontal tubes have projections arranged alternately at intervals on the bottom of the water tank.

In a preferred embodiment of the present application, the reactor body can be made of one or more materials selected from the group consisting of cement, plastic, stainless steel, and fiber-reinforced plastics(FRP).

In a preferred embodiment of the present application, the reactor body has a depth ranging from 20 cm to 40 cm, and the water tank has a shape of circle, square, ellipse, oblong, or square with four transitioning arc corners. The water tank's area can be (8 m−12 m)×(4 m−6 m). The water tank may be closed, or have a top opening, or be a closed tank having an opening with a removable cover.

In a preferred embodiment of the present application, the water tank comprises a pair of opposing side walls, with both ends of each transparent casing passing through the side walls; the openings at the two ends of the transparent casing are oriented towards the exterior, and there is sealed connection between the transparent casing and the side walls of the water tank.

In a preferred embodiment of the present application, a plurality of holes are provided on the pair of side walls, which are arranged in pairs on the side walls, and the transparent casings are installed in the corresponding pairs of holes on the opposite sides of the side walls.

In a preferred embodiment of the present application, the transparent casings are either acrylic tubes or glass tubes, positioned both horizontally and vertically within the water tank.

In a preferred embodiment of the present application, two adjacent ones of the transparent casings are aligned both vertically and horizontally, and arranged at intervals in the water tank; or two adjacent ones of the transparent casings are within the same vertical plane in the vertical direction, and are arranged in a staggered manner horizontally.

In a preferred embodiment of the present application, the reactor body is made of cement or fiber-reinforced plastics, and the transparent casing is pre-embedded in a suitable position during the manufacturing process so that the reactor body and the transparent casing form a one-piece structure; each of the transparent casings extends through two opposite side walls of the water tank with the openings at the two ends facing outside, and the connections between the transparent casing and the side walls of the water tank are watertight to prevent the culture solution in the water tank from leaking out through the installment position of the transparent casing.

This structure facilitates the maintenance and repair of the LED light strips, circuit wiring and connection to power supplies, as well as the installment and dismantlement of radiating tube.

In a preferred embodiment of the present application, when the reactor body is made of stainless steel or plastic material, a plurality of holes for installing the transparent casings are preserved during the manufacturing process of the reactor body. In another embodiment, the holes are excavated in the side walls after the manufacturing is completed. Each of the transparent casings passes through the holes in two opposite side walls of the water tank in such a way that the openings at the two ends of the transparent casing face outwards, and the connections between the holes and the transparent casing coated with a structural sealant to prevent the culture solution in the water tank from leaking out through the installment position of the transparent casing.

In a preferred embodiment of the present application, the light source device further comprises a radiating tube and supporting brackets, wherein the radiating tube is fixed in the core of the transparent casings through the supporting brackets, and the LED light strips are fixed on the outer wall of the radiating tube. The radiating tube is a lengthy tube with one end entering one transparent casing, traversing into another adjacent transparent casing, emerging from it, and repeats this process until all transparent casings are connected; and the radiating tube has circulating refrigerant there inside to cool down the LED light strips effectively.

In a preferred embodiment of the present application, the radiating tube traverses through each of the transparent casings in S shape, either horizontally or vertically.

In particular, the LED light strips can operate intermittently with adjustable light intensity.

In a preferred embodiment of the present application, the high-pressure rinsing and disinfecting device further comprises a frame with inner hollow tubes or a middle thick tube, and the horizontal tubes are interconnected with the frame or the middle thick tube.

In a preferred embodiment of the present application, the liquid nozzles are 360° rotating liquid nozzles.

In a preferred embodiment of the present application, the reactor body is further equipped with a gas supply device, the gas supply device comprises at least one set of ventilation tubes, wherein one end of each ventilation tube is connected to a gas source, and the ventilation tubes are arranged at the bottom of the water tank with a plurality of gas outlets.

In a preferred embodiment of the present application, the ventilation tubes comprise a closed extending tube which has a shape is consistent with the shape of the bottom of the water tank; the ventilation tubes further comprise a fishbone-shaped tube which comprises a main tube extending along the central axis of the water tank and branch tubes extending to the left and right sides of the main tube, and the branch tubes are interconnected with the main tube; the closed extending tube and the fishbone-shaped tube are equipped with a plurality of gas outlet nozzles at interval and are connected to a high-pressure gas supply source respectively through a gas inlet.

In a preferred embodiment of the present application, the photobioreactor further comprises a temperature-regulating device for the culture medium, which comprises a plurality of heat exchangers, and the heat exchangers are installed in the water tank of the bioreactor body vertically, and are arranged alternately at intervals with the transparent casings and the horizontal tubes; the heat exchanger has an S shape in the same plane with the induction of heat medium or refrigerant inside; and the inlets and outlets of the heat exchange tube are respectively located on the exterior of two opposite side walls of the water tank.

When the hybrid photobioreactor of the present application for microalgae cultivation, the water tank is used for containing culture solution and algal strains, and the light source device can provide illumination to the algae beneath the culture surface when natural light is insufficient or at night, so that all algal cells within the culture can receive sufficient light for photosynthesis. In particular, the light source device includes a radiating tube and LED light strips arranged within the transparent casings, and the transparent casings are immersed below the surface of the algal culture, allowing the device to provide light for the algal culture beneath the surface.

The LED light strips are fixed on the side wall of the radiating tube with circulating refrigerant inside, so as to facilitate the dissipation of heat generated by the light emitting action of the LED light strips thus preventing the accumulation of heat and ensuring the algal culture remains at an optimal temperature for the growth. The transparent casings are affixed on the side wall of the water tank of the reactor body with their openings facing outwards. This arrangement allows for the straightforward installation of structures, such as LED light strips, directly into the transparent casings, simplifying tasks such as dismantling and power connection.

The high-pressure rinsing and disinfecting device includes a plurality of horizontal tubes supported by vertical tubes underneath, and each vertical tube extends toward the bottom of the water tank. The horizontal tubes are connected to a general liquid inlet, which is connected to high-pressure clean water, sterilizing water or sterilizing steam that can be used to clean and disinfect the inner wall of the water tank, the outer wall of the transparent casings, and other components, which is conducive to the next round of culture for microalgae. In addition, when the culture solution evaporates and depletes, the high-pressure rinsing and disinfection device can also be used to introduce culture solution, which can also serve to agitate the microalgae being cultured. The transparent casings and a row of a plurality vertical tubes beneath each horizontal tube are interspersed with each other and arranged at intervals.

The present application further provides a gas supply device, which includes ventilation tubes positioned at the bottom of the water tank and can supply the algal culture with necessary CO₂ (or air) crucial for the growth of microalgae. The air bubbles' floating up can help to stir the algal culture, disperse the culture medium evenly, and prevent the algal culture from settling, allowing the algal cells in the water tank to grow and reproduce evenly.

The present application further provides a temperature adjustment device, including several heat exchangers formed by bending heat exchange tubes. These heat exchangers are also installed within the water tank, preferably in the width direction, with the inlets and outlets of the heat exchangers positioned outside the water tank. The arrangement involves interspersing and spacing the heat exchangers, the transparent casings, and a row of vertical tubes below each horizontal tube at intervals. The above-mentioned transparent casings, heat exchange tubes, horizontal tubes, vertical tubes, and the like, partition the water tank into several interconnected small compartments, facilitating the circulation of the algal culture. Accordingly, the photobioreactor of the present application effectively combines the characteristics of water tank reactors, raceway pond reactors, vertical tubular reactors, and horizontal tubular reactors, making it the designation of a “hybrid” reactor.

The hybrid photobioreactor of the present application seamlessly integrates sunlight with artificial light sources, open-type with closed-type, sterilization with culture in an open environment, and it has a compact specific surface area, low manufacturing cost per unit volume, low energy consumption, substantial culture volume, and high solar energy utilization, and enables precise control of the culture temperature, along with easy cleaning and disinfection.

### BRIEF DESCRIPTION OF THE DRAWINGS

FIG. 1 is a schematic diagram of the reactor body of the hybrid photobioreactor of the present application.

FIG. 2 is a schematic diagram of a light source device installed on the reactor body in an embodiment of the present application.

FIG. 3 is a partial enlarged view of the light source device shown in FIG. 2.

FIG. 4 is a schematic diagram of a light source device installed on the reactor body in another embodiment of the present application.

FIG. 5 is a schematic diagram of the way in which a radiating tube in the light source device runs through the transparent casing in an embodiment of the present application.

FIG. 6 is a partial enlarged view of FIG. 5.

FIG. 7 is a schematic diagram of the way in which a radiating tube in the light source device runs through the transparent casing in another embodiment of the present application.

FIG. 8 is a schematic structural diagram of a gas supply device in an embodiment of the present application.

FIG. 9 is a schematic structural diagram of a high-pressure rinsing and disinfecting device in an embodiment of the present application.

FIG. 10 is a schematic diagram of the high-pressure rinsing and disinfecting device of FIG. 9 installed in a reactor body.

FIG. 11 is a schematic structural diagram of a high-pressure rinsing and disinfecting device in another embodiment of the present application.

FIG. 12 is a schematic side structural diagram of a temperature regulating device in a water tank in an embodiment of the present application.

FIG. 13 is a schematic end-side structural diagram of the temperature regulating device in a water tank shown in FIG. 12.

### DETAILED DESCRIPTION OF THE EMBODIMENTS

In order to better explain the present application and facilitate understanding, the present application will be described in detail below through specific embodiments in conjunction with the accompanying drawings.

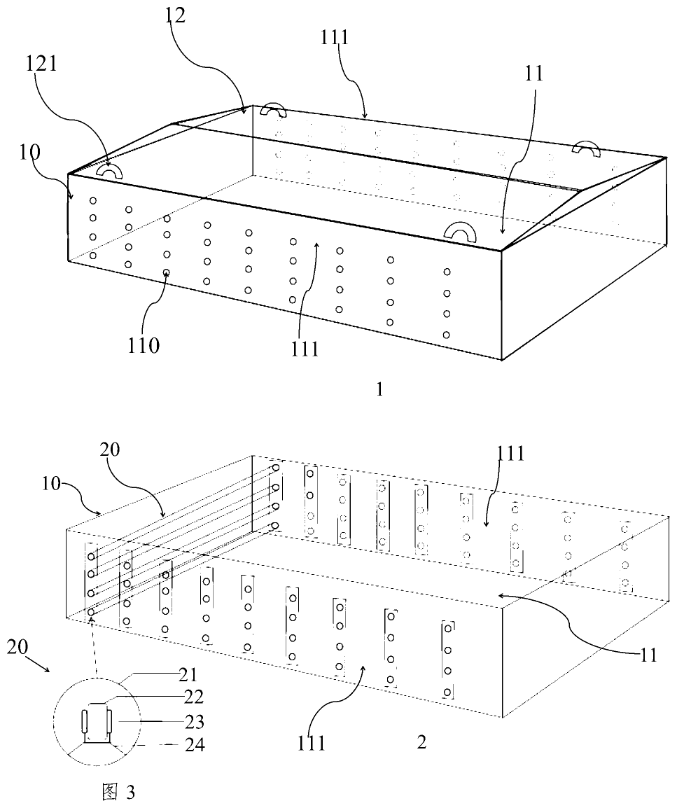

FIG. 1 is a schematic diagram illustrates the reactor body 10 of the hybrid photobioreactor of the present application. The reactor body 10 has an internal water tank 11, which may contain microalgal cell cultures and be equipped with other components. In particular, the reactor body 10 may be made of one or more materials selected from the group consisting of cement, plastic, stainless steel, and fiber-reinforced plastics. The shape of the water tank 11 is versatile and not limited, and may be one or more shapes selected from the group consisting of circle, square, ellipse, oblong, or square with four transitioning arc corners. Specifically, the shape of the water tank 11 can be tailored based on local conditions. The depth of the water tank 11 is from 20 cm to 40 cm. If it is rectangular, oblong or square with four transitioning arc corners, the area may be adjusted within the range of (8 m−12 m)×(4 m-6 m). The water tank 11 may be closed to isolate from the external environment, or possess a top opening to receive natural light, or have an opening with a removable cover, making the form of the water tank 11 more versatile and diverse. Please refer to FIG. 1, it is a water tank 11 with a top opening, and two lids 12 are provided on the top, and the lids 12 are equipped with handles 121, which is convenient for opening.

Please refer to FIG. 1, in this specific embodiment, the water tank 11 is in rectangular shape with a depth of 28 cm, a length of 10 m, and a width of 5 m. In particular, the water tank 11 includes a pair of side walls 111 opposite to each other, and a plurality of holes 110 are provided on the pair of side walls 111. These holes 110 can be used to accommodate the light source device 20. The holes 110 are arranged in pairs on the side walls 111, that is, one hole 110 is provided on one side wall 111, and a corresponding hole 110 is provided on the corresponding other side wall 111.

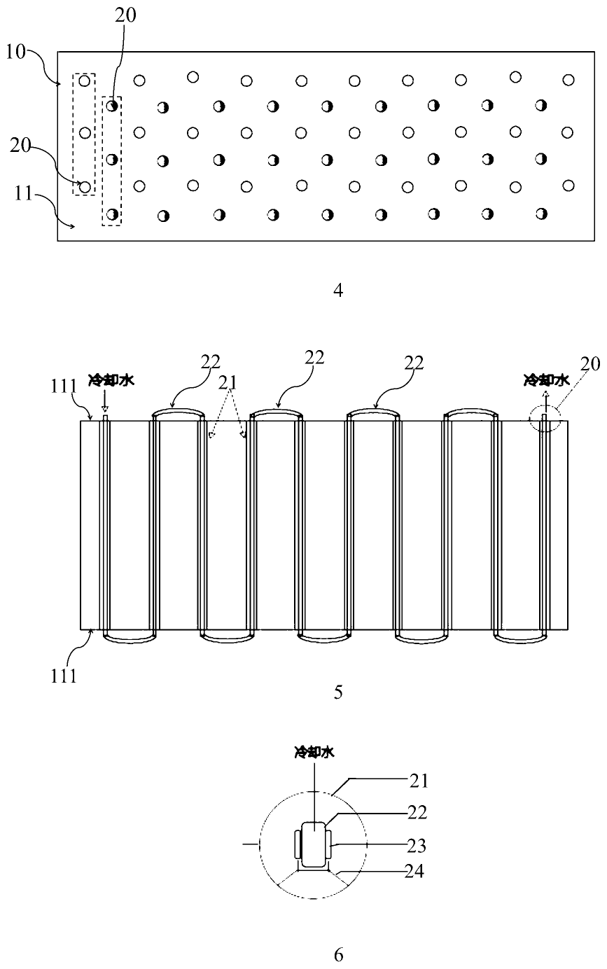

Please refer to FIGS. 2 and 3, the light source device 20 includes transparent casings 21, a radiating tube 22, LED light strips 23 and supporting brackets 24. These transparent casings 21 are positioned within the holes 110 of the side walls 111, and are made of highly transparent material, such as acrylic or glass. The transparent casing 21 is used to house the radiating tube 22, the LED light strips 23 and the supporting brackets 24. In particular, as shown in FIG. 3, the radiating tube 22 runs through the center of the transparent casings 21; the LED light strips 23 are affixed to the outer wall of the radiating tube 22, which, in turn, is secured within the core of the transparent casings 21 through supporting brackets 24. The supporting bracket 24 has a C-shaped supporting seat 241 enveloping the outer wall of the radiating tube 22, and supporting legs 242 (not labeled in the drawing) connected to the supporting seat 241 and fixed to the inner wall of the transparent casing 21 (can also be placed directly in the transparent casing 21). In the embodiment shown in FIG. 2, two adjacent transparent casings 21 are aligned in both vertically and horizontally (in the same plane), arranged at intervals within the water tank 11. The LED light strips 23 installed in each transparent casing 21 share the same configuration and specifications.

FIG. 4 is a schematic diagram of one side of a water tank in another embodiment of the present application. In this embodiment, two adjacent transparent casings 21 are in the same vertical plane in the vertical direction, but are arranged in a staggered manner in the horizontal direction. The LED light strips 23 installed in different transparent casings 21 can have different configurations and specifications. For example, some LED light strips in the transparent casings 21 emit red light, some emit blue light, etc. The LED light strips can be configured to have different light intensities and different powers.

The installation method of the transparent casings 21 on the side walls 111 of the water tank 11 is versatile, however, in this approach, both ends of each transparent casing 21 pass through the side walls 111, with the openings of the two ends of the transparent casing 21 facing outward. It is crucial to ensure that the connections between the transparent casing 21 and the side walls 111 of the water tank 11 are watertight. This structure can not only safeguard the LED light strip 23 from potential malfunctions caused by water ingress but also guarantees that the LED light strips 23 are immersed in the culture medium within the water tank 11 (serving as an integrated light source for the culture medium) and directly illuminate the algal cells to improve the light utilization efficiency. More importantly, this structure is highly conducive to the maintenance and repair of the LED light strips 23, circuit wiring and connection to power supplies, replacement, installation and dismantlement of radiating tube 22.

For example, when the reactor body 10 is made of cement or fiber-reinforced plastics, the transparent casing 21 may be pre-embedded in a suitable position during the manufacturing process. Subsequently, when cement is poured, the reactor body 10 and the transparent casing 21 amalgamate into a single-piece structure. Each transparent casing 21 runs through the two opposite side walls 111 of the water tank 10, and anti-seepage treatment is performed at the connection points between each transparent casing 21 and the side walls 111. Alternatively, when the reactor body 10 is made of stainless steel or plastic material, holes 110 for installing the transparent casings 21 may be predetermined during the manufacturing process of the reactor body 10. Alternatively, the holes 110 may be excavated in the side walls 111 after the manufacturing is completed. Once the transparent casings 21 are installed in a plurality of holes 110 on the two opposite side walls, a structural sealant is applied to the spaces between the holes 110 and the transparent casings 21 to avoid water seepage.

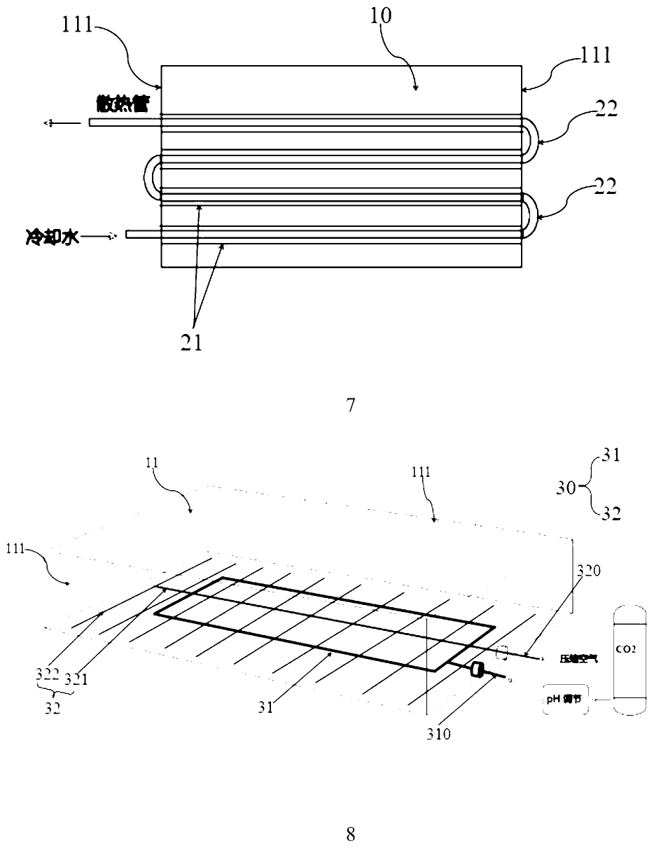

FIG. 5 is a schematic top view of the water tank 11, showing how the radiating tube 22 in the light source device 20 runs through the transparent casings 21. FIG. 6 is a partial enlarged view of FIG. 5. In this embodiment, the radiating tube 22 runs through a plurality of transparent casings 21 in the same horizontal plane. Specifically, one end of the radiating tube 22 runs through one transparent casing 21, into another adjacent transparent casing 21 in the same horizontal plane, and then continues to run into the third adjacent transparent casing 21 in the same horizontal plane until all the transparent casings 21 in the horizontal plane are run through. One end of the radiating tube 22 is the inlet for refrigerant, and the other end is the outlet. The refrigerant is preferably cooling water.

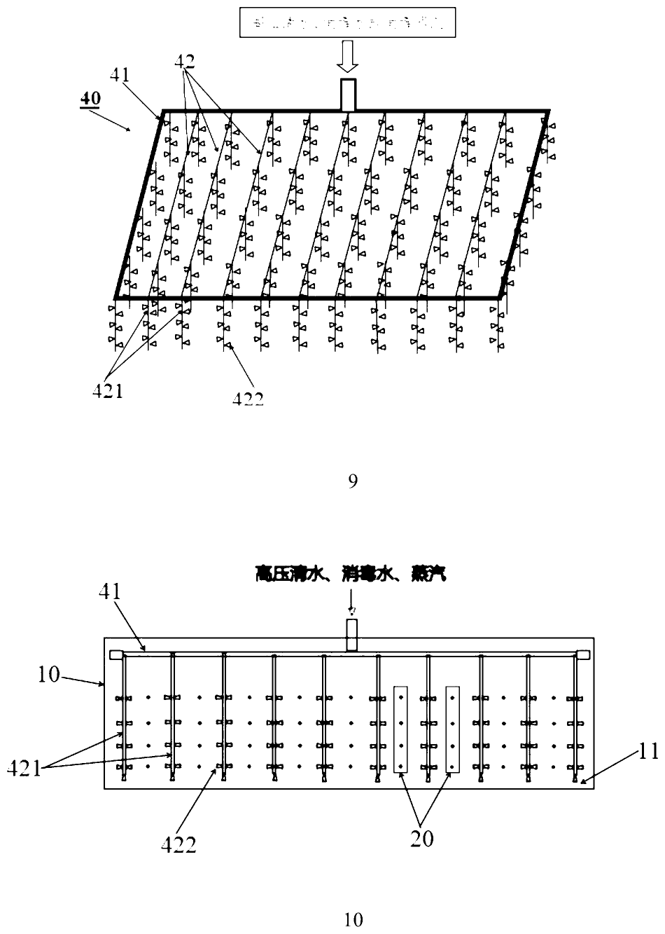

FIG. 7 is a schematic end-side structural diagram of the water tank 11, showing how the radiating tube 22 in the light source device 20 traverses through the transparent casings 21. In this embodiment, the radiating tube 22 runs through a plurality of transparent casings 21 within the same vertical plane. Specifically, one end of the radiating tube 22 runs through one transparent casing 21, extends into another adjacent transparent casing 21 within the same vertical plane. This sequence continues until the radiating tube 22 has traversed through all the transparent casings in the same vertical plane. One end of the radiating tube 22 acts as the inlet for refrigerant, while the other end serves as the outlet. The refrigerant is preferably cooling water.

Alternatively, a radiating tube 22 can traverse through all the transparent casings 21 in a specific order. It may follow a sequential path, running through each transparent casing 21 on the bottom layer first; after reaching one end of the water tank 11, it can proceed to traverse through each transparent casing 21 on the second layer in sequence; this process continues until reaching one end of the water tank 11, and repeats for subsequent layers until the radiating tube 22 has traversed through all the transparent casings 21 provided on the water tank 11

During installment, in order to facilitate the assembly of the LED light strips 23, the supporting brackets 24 can be pre-set in each transparent casing 21, and the C-shaped support seat 241 of the supporting bracket 24 has an opening. The LED light strips 23 are affixed on both sides of the radiating tube 22 at regular intervals, bonded with thermally conductive adhesive. A curved section of the radiating tube 22 outside the water tank 11 is intentionally left without LED light strips. Then, the radiating tube 22 bonded with the LED light strips 23 is installed by being made to traverse through each transparent casings 21 in sequence according to the abovementioned methods or other methods, and then it is fixed on the C-shaped supporting seats 241. Finally, the supporting seats 241 are clamped with tools like pliers, ensuring the fixation of the radiating tube 22 and the LED light strips 23.

FIG. 8 is a schematic structural diagram of the gas supply device 30, which is provided at the bottom of the water tank 11. The gas supply device 30 includes a closed extending tube 31 whose shape aligns with the bottom of the water tank 11. If the bottom of the water tank 11 is square, the closed extending tube 31 forms a square shape and is connected to the high-pressure CO₂ gas source through the gas inlet 310. The closed extending tube 31 is equipped with a plurality of dispersed gas outlets separately, which may introduce CO₂ bubbles into the water tank 11 to enhance the autotrophic photosynthesis of microalgae, and can also adjust the pH of the algal culture and agitate the algal culture to prevent algal cells from settling at the bottom of the water tank 11. The gas supply device 30 also includes a fish bone-shaped tube 32. The fish bone-shaped tube 32 includes a main tube 321 extending along the central axis of the water tank and branch tubes 322 extending to the left and right sides of the main tube 321, wherein the branch tubes 322 are connected with the main tube 321. The fish bone-shaped tube 32 is also equipped with several gas outlet nozzles provided in the middle and top of each branch tube 322. The main tube 321 is connected to a high-pressure gas supply source, such as an air source, through the gas inlet 320, which mainly serves as an agitator.

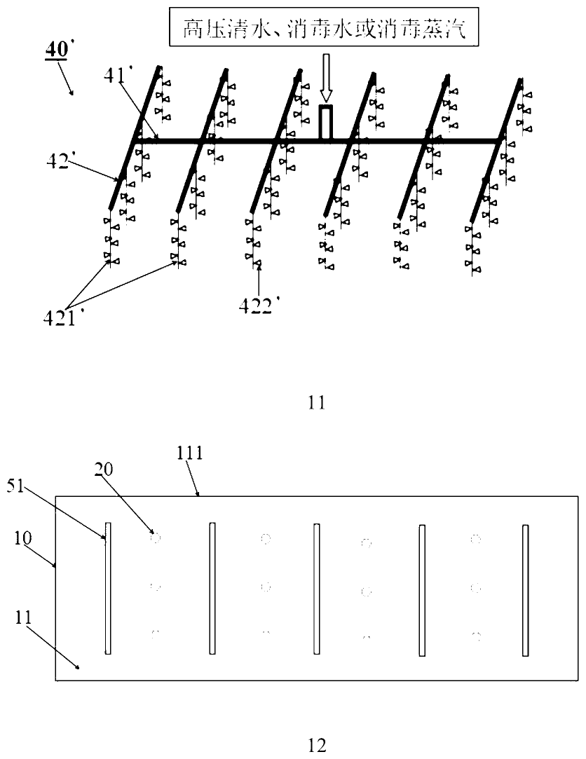

FIG. 9 is a schematic structural diagram of a high-pressure rinsing and disinfecting device 40 in a preferred embodiment of the present application. The high-pressure rinsing and disinfecting device 40 is installed within the water tank 11. The high-pressure rinsing and disinfecting device 40 includes a frame 41 with hollow tubes therein. A plurality of horizontal tubes 42 are provided in the frame 41, and the horizontal tubes 42 are connected with the hollow tubes of the frame 41, and each horizontal tube 42 interfaces with several vertical tubes 421 below, forming a comb-shaped structure with the connected vertical tubes 421. As shown in FIG. 9, a plurality of liquid nozzles 422 are provided on the wall of each vertical tube 421 from top to bottom. The lower end of the vertical tube 421 extends into the water tank 11 and is situated close to the bottom of the water tank 11. There is a general liquid inlet 410 (not labeled in the drawings) on the frame 41. Each horizontal tube 42 can be connected to the general liquid inlet 410 which is connected to high-pressure clean water, sterilizing water or sterilizing steam.

Please refer to FIG. 10, these vertical tubes 421 are located between two adjacent vertical rows of transparent casings 21; in other words, the projections of the transparent casings 21 and the horizontal tubes 42 on the bottom of the water tank 11 are arranged alternately at intervals.

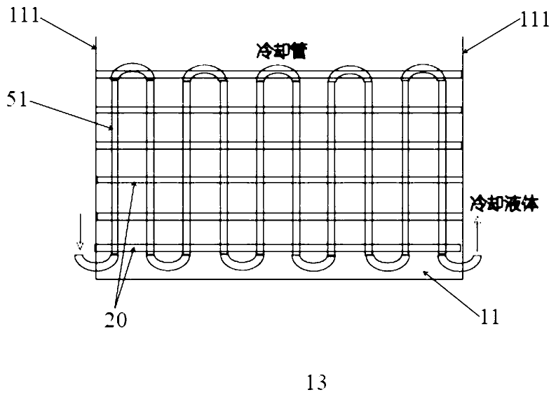

In another embodiment of the present application, as shown in FIG. 11, the high-pressure rinsing and disinfecting device 40′ may not be equipped with a frame 41, but may comprise a middle thick tube 41′, several horizontal tubes 42′ connected on both sides, and several vertical tubes 421′ arranged vertically below the horizontal tube 42′. The wall of each vertical tube 421′ is equipped with a plurality of liquid nozzles 422′ from top to bottom. The lower end of the vertical tube 421′ extends into the water tank 11. Similarly, the middle thick tube 41′ is equipped with a general liquid inlet 410′ (not labeled in the drawings) which is connected to high-pressure clean water, sterilizing water or sterilizing steam. Similarly, the projections of the transparent casings 21 and the horizontal tubes 42′ on the bottom of the water tank 11 are arranged alternately at intervals.

The above-mentioned liquid nozzles 422 and 422′ can both be set to be 360° rotating liquid nozzles. The structure of the above-mentioned high-pressure rinsing and disinfecting device 40 (40′) allows for the rapid cleaning and disinfection of the inner wall of the water tank 11, preparing it for the subsequent culture cycles. In addition, when the culture medium evaporates and decreases, it can also be replenished through the high-pressure rinsing and disinfecting device 40, providing strong agitation to the algal culture during replenishment.

Please refer to FIGS. 12-13, the photobioreactor of the present application is further equipped with temperature-regulating device for culture medium. Specifically, as shown in FIG. 12, it includes a plurality of heat exchangers 51. These heat exchangers 51 are vertically installed in the water tank 11 at intervals, and each interval aligns with the locations of the transparent casings 21 and the vertical tubes 421 (421′) As shown in FIG. 13, each heat exchanger 51 comprises a heat exchange tube bent into an S shape within the same plane. The heat medium or refrigerant is introduced into the heat exchange tube, and the inlets and outlets of the heat exchange tube are respectively located on the exterior of two opposite side walls 111 of the water tank 11. Each heat exchanger 51 is connected to the heating medium or refrigerant individually, or all the heat exchangers 51 are linked end-to-end outside the water tank 11 to connect the circulating heating medium or refrigerant, thereby effectively regulating the temperature of the algal culture in the water tank 11.

The above-mentioned transparent casings 21, heat exchange tubes, horizontal tubes 42 (42′), vertical tubes 421 (421′), and the like, partition the water tank into several interconnected small compartments, facilitating the circulation of the algal culture. Accordingly, the photobioreactor of the present application effectively combines the characteristics of water tank reactors, raceway pond reactors, vertical tubular reactors, and horizontal tubular reactors, making it the designation of a “hybrid” reactor. The hybrid photobioreactor of the present application seamlessly integrates sunlight with artificial light sources, open-type with closed-type, sterilization with culture in an open environment, and it has a compact specific surface area, low manufacturing cost per unit volume, low energy consumption, substantial culture volume, and high solar energy utilization, and enables precise control of the culture temperature, along with easy cleaning and disinfection.

Finally, it should be noted that the above embodiments are only used to illustrate the technical solution of the present application, rather than to limit it. Although the present application has been described in detail with reference to the above embodiments, those of ordinary skill in the art should understand that: the technical solutions described in the foregoing embodiments can still be modified, or some or all of the technical features can be equivalently replaced; and these modifications or replacements will not cause the essence of the corresponding technical solutions to deviate from the scope of the technical solutions of the embodiments of the present application.

What is claimed is:

1. A hybrid photobioreactor, comprising:
a reactor body which is equipped with a water tank configured to accommodate microalgal cell cultures, a light source device, and a high-pressure rinsing and disinfecting device;
wherein the light source device comprises a plurality of transparent casings and corresponding LED light strips located therein, and the transparent casings are arranged in parallel at intervals within the water tank;
the high-pressure rinsing and disinfecting device comprises a plurality of horizontal tubes and corresponding vertical tubes located thereunder, and each of the horizontal tubes forms a comb-shaped structure with the corresponding vertical tube connected thereto; each of the vertical tubes has a plurality of liquid nozzles provided on the wall thereof from top to bottom, and has a lower end close to a bottom of the water tank; the plurality of horizontal tubes are connected to a general liquid inlet which is connected to high-pressure clean water, sterilizing water or sterilizing steam; the transparent casings and the horizontal tubes have projections arranged alternately at intervals on the bottom of the water tank.

2. The hybrid photobioreactor of claim 1, wherein the reactor body is made of one or more materials selected from the group consisting of cement, plastic, stainless steel, and fiber-reinforced plastics; the reactor body has a depth of 20 cm-40 cm, and has a shape of circle, square, ellipse, oblong, or square with four transitioning arc corners; and the water tank is closed, or has a top opening, or has an opening with a removable cover.

3. The hybrid photobioreactor of claim 1, wherein the water tank comprises a pair of side walls opposite to each other and both ends of each of the transparent casings run through the side walls; the two ends of the transparent casing have openings facing outside of the water tank, and the transparent casings and the side walls of the water tank have connections which are sealed.

4. The hybrid photobioreactor of claim 3, wherein the pair of side walls each has a plurality of holes, which are arranged in pairs on the side walls, and the transparent casings are installed in the holes of the side walls opposite to each other.

5. The hybrid photobioreactor of claim 4, wherein the transparent casings are acrylic tubes or glass tubes which are located in the horizontal and vertical directions of the water tank.

6. The hybrid photobioreactor of claim 5, wherein two adjacent ones of the transparent casings are aligned both vertically and horizontally and arranged at intervals in the water tank; alternatively, two adjacent ones of the transparent casings are within a same vertical plane in the vertical direction, and are arranged in a staggered manner horizontally.

7. The hybrid photobioreactor of claim 1, wherein the light source device further comprises a radiating tube and a supporting bracket, the radiating tube is fixed in a center of the transparent casings through the supporting bracket, and the LED light strips are fixed on an outer wall of the radiating tube; the radiating tube is a lengthy tube passing through all the transparent casings sequentially, which has circulating refrigerant inside to cool down the LED light strips.

8. The hybrid photobioreactor of claim 1, wherein the radiating tube traverses through all the transparent casings in S shape horizontally or vertically.

9. The hybrid photobioreactor of claim 1, wherein the LED light strips operate intermittently with adjustable light intensity.

10. The hybrid photobioreactor of claim 1, wherein the high-pressure rinsing and disinfecting device further comprises a frame with inner hollow tubes or a middle tube, and the horizontal tubes are interconnected with the frame or the middle tube.

11. The hybrid photobioreactor of claim 1, wherein the liquid nozzles are 360° rotating liquid nozzles.

12. The hybrid photobioreactor of claim 1, wherein the reactor body is further equipped with a gas supply device, the gas supply device comprises at least a set of ventilation tubes with one end connected to a gas source, and the ventilation tubes are arranged at the bottom of the water tank with a plurality of gas outlets.

13. The hybrid photobioreactor of claim 12, wherein the ventilation tubes comprise a closed extending tube having a shape matching with that of the bottom of the water tank; the ventilation tubes further comprise a fishbone-shaped tube with a main tube extending along a central axis of the water tank and branch tubes extending to left and right sides of the main tube, and the branch tubes are interconnected with the main tube; the closed extending tube and the fishbone-shaped tube are equipped with a plurality of gas outlet nozzles at interval and are connected to a high-pressure gas supply source respectively through a gas inlet.

14. The hybrid photobioreactor of claim 1, wherein the photobioreactor further comprises a temperature regulating device for culture liquid which comprises a plurality of heat exchangers which are installed in the water tank of the bioreactor body vertically, and are arranged alternately at intervals with the transparent casings and the horizontal tubes.

15. The hybrid photobioreactor of claim 14, wherein the heat exchanger is an S-shaped heat exchange tube in a same plane; the heat exchange tube has heat medium or refrigerant introduced there into; and the inlets and outlets of the heat exchange tube are respectively located outside of two opposite side walls of the water tank.
